(19) United States
(12) Patent Application Publication
(10) Pub. No.: US 2026/0315521 A1
(43) Pub. Date: Oct. 15, 2026

(54) THERAPEUTIC FORMULATIONS FOR KIDNEY DISEASE COMPRISING REPAGERMANIUM OR PROPAGERMANIUM AND/OR AT1R BLOCKERS

(71) Applicant: DIMERIX BIOSCIENCE PTY LTD, Fitzroy, Victoria (AU)

(72) Inventors: Carl William WHITE, Niddrie (AU); Robert Pearson SHEPHERD, Upwey (AU)

(73) Assignee: DIMERIX BIOSCIENCE PTY LTD, Fitzroy, Victoria (AU)

(21) Appl. No.: 19/482,619

(22) PCT Filed: Apr. 30, 2024
(86) PCT No.: PCT/AU2024/050416

§ 371 Date: Nov. 7, 2025

(30) Foreign Application Priority Data
May 12, 2023 (AU) 2023901450

(51) Int. Cl. A61K 31/80; A61K 31/4178; A61K 31/4184; A61K 31/422; A61K 31/513; A61P 13/12

(52) U.S. Cl. CPC A61K 31/80; A61K 31/4178; A61K 31/4184; A61K 31/422; A61K 31/513; A61P 13/12

(57) ABSTRACT

The present invention relates to therapeutic formulations adapted to deliver a daily dose of from about 200 mg to 280 mg of repagermanium or propagermanium, therapeutic formulations adapted to deliver a daily dose of a therapeutically effective amount of an ATiR blocker that has an affinity for the CCR2-AT1R complex that is the same or greater than the ATiR blocker's affinity for AT,R only, and combinations of these actives. These formulations and combinations are used for the treatment of kidney diseases.

### TECHNICAL FIELD

The present disclosure relates to therapeutic formulations comprising repagermanium or propagermanium and methods of using the same and therapeutic formulations comprising an AT₁R blocker and methods of using the same. The repagermanium or propagermanium may be delivered in a therapeutic combination regimen with an AT₁R blocker.

### BACKGROUND ART

Therapies for diseases such as kidney disease and respiratory disease based on a chemokine receptor 2 (CCR2) pathway inhibitor or an AT₁R blocker, optionally in a therapeutic combination regimen, are known.

Repagermanium or propagermanium may be used as a CCR2 pathway inhibitor. Repagermanium and propagermanium have previously been described in the literature as polymeric compounds. The drug substance 3-[(2-carboxyethyl-oxogermyl)oxy-oxogermyl]propanoic acid exhibits polymorphism and can exist in different solid forms. However, the present inventors have not observed any pharmacological effects of polymerisation of repagermanium during manufacture or testing of the drug substance.

Polymorphs of 3-[(2-carboxyethyl-oxogermyl)oxy-oxogermyl] propanoic acid include repagermanium, propagermanium, proxigermanium, Ge-132, germanium sesquioxide, 2-carboxyethylgermasesquioxane, SK-818, bis(2carboxyethylgermanium) sesquioxide.

The two most highly characterised solid forms, repagermanium and propagermanium share a common core structure (Mizuno, et al, 2015). These two drug substances have different International Nonproprietary Names (INNs), the INN for repagermanium being INN #6259 and propagermanium being INN #6260. They are comprised of different packing of the monomeric molecule 3-[(2-carboxyethyl-oxogermyl)oxy-oxogermyl] propanoic acid, varying in the structural units coordinated by H bonds. X-ray diffraction has revealed that repagermanium has an infinite sheet structure which is clearly distinguishable from the propagermanium polymeric ladder-shaped structure of a concatenated eight-membered ring composed of Ge-O bonds. In solid form, repagermanium and propagermanium differ; however, in solution they are identical as the structural units are dissolved.

The angiotensin type 1 receptor (AT₁R, AT1R, Angiotensin II receptor type 1) is a G protein-coupled receptor. In respect of their effect on AT₁R blockage, AT₁R blockers (also known as ARBs) are traditionally seen to be therapeutically interchangeable despite structural differences.

However, dosing regimens for repagermanium or propagermanium and AT₁R blockers are still to be definitively established for various diseases. There is a need to establish suitable dosing regimens for repagermanium or propagermanium and AT₁R blockers; or at least the provision of new dosing regimens to compliment the previously known dosing regimens. The present disclosure seeks to provide an improved or alternative dosing regimens for repagermanium or propagermanium and AT₁R blockers. The repagermanium or propagermanium may be delivered in a therapeutic combination regimen with an AT₁R blocker.

The previous discussion of the background art is intended to facilitate an understanding of the present disclosure only. The discussion is not an acknowledgement or admission that any of the material referred to is or was part of the common general knowledge as at the priority date of the application.

### SUMMARY OF INVENTION

The present disclosure provides a therapeutic formulation adapted to deliver a daily dose of from about 200 mg to 280 mg repagermanium or propagermanium.

The present disclosure further provides a therapeutic formulation adapted to deliver a daily dose of a therapeutically effective amount of an AT₁R blocker that has an affinity for the CCR2-AT₁R complex that is the same or greater than the AT₁R blocker's affinity for AT₁R only.

The present disclosure further provides a therapeutic formulation adapted to deliver daily dose of: (a) a therapeutically effective amount of repagermanium or propagermanium; and (b) a therapeutically effective amount of an AT₁R blocker that has an affinity for the CCR2-AT₁R complex that is the same or greater than the AT₁R blocker's affinity for AT₁R only.

Optionally, the daily dose of repagermanium or propagermanium is from about 200 mg to 280 mg.

Optionally, the AT₁R blocker is chosen from the list consisting of: candesartan, eprosartan, fimasartan, irbesartan, losartan, olmesartan, telmisartan, valsartan and sparsentan.

Optionally, the daily dose of the AT₁R blocker is from about 2 mg to 800 mg.

The present disclosure further provides a dosage regimen comprising administering a daily dose of from about 200 mg to 280 mg repagermanium or propagermanium.

The present disclosure further provides a dosing regimen comprising administering a daily dose of a therapeutically effective amount of an AT₁R blocker that has an affinity for the CCR2-AT₁R complex that is the same or greater than the AT₁R blocker's affinity for AT₁R only.

The present disclosure further provides a dosing regimen comprising administering a daily dose of: (a) a therapeutically effective amount of repagermanium or propagermanium; and (b) a therapeutically effective amount of an AT₁R blocker that has an affinity for the CCR2-AT₁R complex that is the same or greater than the AT₁R blocker's affinity for AT₁R only.

The present disclosure further provides a method for treatment, amelioration or prevention of a kidney disease, said method comprising the step of administering to a subject: (a) a therapeutic formulation adapted to deliver from about 200 mg to 280 mg repagermanium or propagermanium per day.

The present disclosure further provides a method for treatment, amelioration or prevention of a kidney disease, said method comprising the step of administering to a subject: (a) a therapeutically effective amount of an AT₁R blocker that has an affinity for the CCR2-AT₁R complex that is the same or greater than the AT₁R blocker's affinity for AT₁R only.

The present disclosure further provides a method for treatment, amelioration or prevention of a kidney disease, said method comprising the step of administering to a subject: (a) a therapeutically effective amount of repagermanium or propagermanium per day; and (b) a therapeutically effective amount of an AT₁R blocker that has an affinity for the CCR2-AT₁R complex that is the same or greater than the AT₁R blocker's affinity for AT₁R only.

The present disclosure further provides for the use of a daily dose of from about 200 mg to 280 mg repagermanium or propagermanium for the manufacture of a formulation for the treatment, amelioration or prevention of a kidney disease in a subject.

The present disclosure further provides for the use of a daily dose of from about 200 mg to 280 mg repagermanium or propagermanium for the treatment, amelioration or prevention of a kidney disease in a subject.

The present disclosure further provides for the use of a therapeutically effective amount of an AT₁R blocker that has an affinity for the CCR2-AT₁R complex that is the same or greater than the AT₁R blocker's affinity for AT₁R only for the manufacture of a formulation for the treatment, amelioration or prevention of a kidney disease in a subject.

The present disclosure further provides for the use of a therapeutically effective amount of an AT₁R blocker that has an affinity for the CCR2-AT₁R complex that is the same or greater than the AT₁R blocker's affinity for AT₁R only for the treatment, amelioration or prevention of a kidney disease in a subject.

The present disclosure further provides for the use of (a) a therapeutically effective amount of repagermanium or propagermanium, and (b) a therapeutically effective amount of an AT₁R blocker that has an affinity for the CCR2-AT₁R complex that is the same or greater than the ATAR blocker's affinity for AT₁R only for the manufacture of a formulation for the treatment, amelioration or prevention of a kidney disease in a subject.

The present disclosure further provides for the use of (a) a therapeutically effective amount of repagermanium or propagermanium, and (b) a therapeutically effective amount of an AT₁R blocker that has an affinity for the CCR2-AT₁R complex that is the same or greater than the ATAR blocker's affinity for AT₁R only for the treatment, amelioration or prevention of a kidney disease in a subject.

The present disclosure further provides a therapeutically effective amount of repagermanium or propagermanium for use in a formulation for the treatment, amelioration or prevention of a kidney disease in a subject, wherein the repagermanium is administered to the subject concurrently or sequentially with a therapeutically effective amount of an AT₁R blocker that has an affinity for the CCR2-AT₁R complex that is the same or greater than the AT₁R blocker's affinity for AT₁R only.

The present disclosure further provides a therapeutically effective amount of an AT₁R blocker that has an affinity for the CCR2-AT₁R complex that is the same or greater than the AT₁R blocker's affinity for AT₁R only for use in a formulation for the treatment, amelioration or prevention of a kidney disease in a subject, wherein the at least one AT₁R blocker is administered to the subject concurrently or sequentially with repagermanium or propagermanium.

The present disclosure further provides a kit for the treatment or prevention of a kidney disease in a subject, said kit comprising: (a) a daily dose of from about 200 mg to 280 mg repagermanium or propagermanium; and (b) instructions for use.

The present disclosure further provides a kit for the treatment or prevention of a kidney disease in a subject, said kit comprising: (a) a therapeutically effective amount of an AT₁R blocker that has an affinity for the CCR2-AT₁R complex that is the same or greater than the AT₁R blocker's affinity for AT₁R only; (b) instructions for use.

The present disclosure further provides a kit for the treatment or prevention of a kidney disease in a subject, said kit comprising: (a) a therapeutically effective amount of repagermanium or propagermanium; (b) a therapeutically effective amount of an AT₁R blocker that has an affinity for the CCR2-AT₁R complex that is the same or greater than the AT₁R blocker's affinity for AT₁R only; and (c) instructions for use.

Optionally, the repagermanium or propagermanium is provided at a daily dose of from about 200 mg to 280 mg repagermanium or propagermanium.

Optionally, the AT₁R blocker is chosen from the list consisting of: candesartan, eprosartan, fimasartan, irbesartan, losartan, olmesartan, telmisartan, valsartan and sparsentan.

Optionally, the subject to be treated is human mammal.

Optionally, the kidney disease is a kidney disease associated with proteinuria.

Optionally, the kidney disease is chosen from the list comprising: focal segmental glomerulosclerosis (FSGS; including idiopathic (primary) FSGS, secondary FSGS, genetic FSGS, and any other causes of FSGS), fibrotic disorders in the kidney, Immunoglobulin A nephropathy (IgAN), Alport's Syndrome, chronic kidney disease including chronic kidney disease caused by diabetic nephropathy, renal insufficiency (diabetic and non-diabetic), and renal failure conditions, including diabetic nephropathy, glomerulonephritis, scleroderma, glomerular sclerosis, proteinuria of primary renal disease and renal vascular hypertension.

Optionally, the AT₁R blocker is not EXP3174.

### BRIEF DESCRIPTION OF THE DRAWINGS

Further features of the present disclosure are more fully described in the following description of several non-limiting embodiments thereof. This description is included solely for the purposes of exemplifying the present disclosure. It should not be understood as a restriction on the broad summary, disclosure or description of the disclosure as set out above. The description will be made with reference to the accompanying drawings in which:

FIG. 1 is a graph of the effect of different doses of repagermanium on efficacy of treatment. Efficacy of repagermanium dose displays a ‘bell-shaped’ inhibitory curve.

FIG. 2 is a graph of the correlation plots comparing ARB potency at the AT₁R only versus potency for inhibition of G protein dissociation from co-stimulated CCR2-AT₁R complexes. Cand; Candesartan, CandC; Candesartan cilexetil, Epro; Eprosartan, EXP; EXP3174. Irb; Irbesartan, Fim; Fimasartan*, Los; Losartan, Olm; Olmesartan, OlmM; Olmesartan medoxomil, Spar; Sparsentan, Tel; Telmisartan, Val; Valsartan.

FIG. 3 is a graph of the statistical analysis of EXP3174 potency (pIC50) at the AT₁R only versus potency for inhibition of G protein dissociation from co-stimulated CCR2-AT₁R complexes. ** p<0.01 measured by a one-sample t-test.

FIG. 4 is a graph of the effect of ARB, CCR2 or ARB+CCR2 inhibition on G protein (Gαi1-Luciferase) dissociation from co-stimulated CCR2-AT₁R complexes.

FIG. 5 is a graph of the Correlation plots comparing ARB potency at the AT₁R only versus potency for inhibition of G protein dissociation from co-stimulated CCR2-AT₁R complexes in the presence of repagermanium *. Cand; Candesartan, CandC; Candesartan cilexetil, Epro; Eprosartan, EXP; EXP3174, Fim; Fimasartan*, Irb; Irbesartan, Los; Losartan, Olm; Olmesartan, OlmM; Olmesartan medoxomil, Spar; Sparsentan, Tel; Telmisartan, Val; Valsartan.

### DESCRIPTION OF INVENTION

### Detailed Description of the Invention

Although CCR2 inhibitors and/or AT₁R blockers have been used in a variety of therapeutic settings, there is still much that is not understood about the dosing regimens that provide effective therapeutic outcomes.

### Formulations

The present disclosure provides therapeutic formulations comprising: (i) repagermanium or propagermanium adapted to be delivered at from about 200 mg to 280 mg repagermanium or propagermanium per day; (ii) an AT₁R blocker that has an affinity for the CCR2-AT₁R complex that is the same or greater than the AT₁R blocker's affinity for AT₁R only; or (iii) repagermanium or propagermanium adapted to be delivered at from about 200 mg to 280 mg repagermanium or propagermanium per day and an AT₁R blocker that has an affinity for the CCR2-AT₁R complex that is the same or greater than the AT₁R blocker's affinity for AT₁R only.

### Repagermanium or Propagermanium Formulation

The present inventors have surprisingly found that the dose of repagermanium or propagermanium required for therapeutic efficacy falls within a narrow dosage and treatment regimen.

Repagermanium and propagermanium blocks pathways associated with MCP-1 induced migration, activation of monocytes and chemotactic migration. Repagermanium and propagermanium further blocks the functioning of cellular structures involved in chemotaxis such as microfilaments, microtubules, and intermediate filaments, and surface proteins such as glycosylphosphatidylinositol (GPI)-anchored proteins, and more specifically CD55, CD59 and CD16.

The INN for repagermanium is INN #6259 and propagermanium is INN #6260. Propagermanium has been used as an active agent against chronic hepatitis and has been shown to specifically inhibit in vitro chemotactic migration of monocytes by MCP-1 through a mechanism that seems to require glycosylphosphatidylinositol (GPI)-anchored proteins such as CD55, CD59 and CD16 (Yokochi, S. (2001) Journal of Interferon and Cytokine Research 21:389-398). Additional names for propagermanium and/or repagermanium include 3-[(2-carboxyethyl-oxogermyl)oxy-oxogermyl] propanoic acid, proxigermanium, Ge-132, bis(2-carboxyethylgermanium) sesquioxide (CEGS), 2-carboxyethylgermasesquioxane, SK-818, organic germanium, germanium sesquioxide, 3,3′-(1,3-dioxo-1,3-digermanoxanediyl) bispropionic acid, 3-oxygermylpropionic acid polymer and poly-trans-(2-carboxyethyl) germasesquioxane. In the present disclosure, the term “repagermanium” is defined to include “propagermanium”.

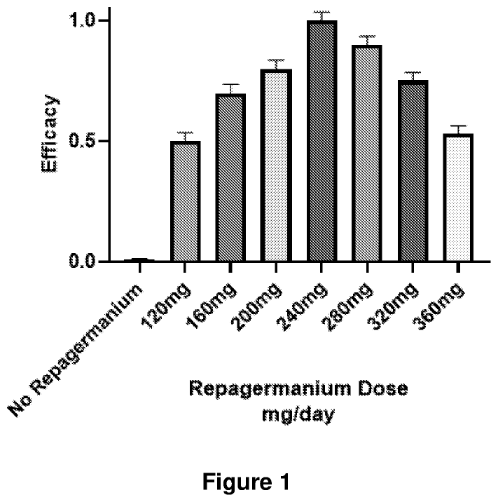

The inventors have found that a dose of, for example 80 mg twice per day, results in the blood concentration of repagermanium or propagermanium falling too low before the next dose to provide a therapeutic effect. Furthermore, contrary to expectations, a dose of, for example 140 mg twice per day, results in a reduced therapeutic effect. Thus, the therapeutic effect of repagermanium or propagermanium is maximised at a moderate dose. This general trend can be seen in FIG. 1.

The present disclosure thus provides a therapeutic formulation adapted to deliver a daily dose of from about 200 mg to 280 mg repagermanium or propagermanium.

The present disclosure further provides a dosage regimen comprising administering a daily dose of from about 200 mg to 280 mg repagermanium or propagermanium.

The repagermanium or propagermanium may be adapted to be delivered at from about 220 mg to 260 mg per day, optionally about 240 mg per day.

The formulation comprising repagermanium or propagermanium may be delivered twice per day (BID). Thus, the formulation can be used in a dosing regimen comprising administering a dose of from about 100 mg to 140 mg repagermanium or propagermanium twice per day. Although a dosing regimen of three times or more per day is contemplated, this is less likely to be acceptable and adhered to by subject receiving the treatment. If the dosing is carried out three times or more per day, the total daily dose should still be from about 200 mg to 280 mg per day, optionally about 240 mg per day.

If the repagermanium or propagermanium is being administered to children under the age of 12, a scaled dose may be administered to achieve the same exposure and pharmacokinetic outcome as the adult dose. The scaled dose may be determined based on weight. The adult dose of from about 200 mg to 280 mg repagermanium per day is based on an average 70 kg adult. For children under the age of 12, the dose may be calculated based on their weight proportion of 70 kg.

The therapeutic formulation comprising repagermanium or propagermanium of the present disclosure has been developed based on pharmacokinetic data, particularly the half-life of the repagermanium and propagermanium in the body and the Cₘₐₓ of repagermanium and propagermanium.

This specific dose and exposure of repagermanium or propagermanium is required for peak therapeutic efficacy based on the reduced chemotaxis of CCR2-expressing cells.

### Angiotensin Type 1 Receptor Blocker Activity

The present inventors have surprisingly found that, although AT₁R blockers are traditionally seen to be interchangeable, there are some exceptions based on the interaction of the AT₁R blocker and the CCR2-AT₁R complex.

The angiotensin type 1 receptor (AT₁R, AT₁R, Angiotensin II receptor type 1) is a G protein-coupled receptor.

The phrase “angiotensin type 1 receptor inhibitor” (also referred to as an angiotensin receptor blocker or ARB) is understood to mean an agent or compound which can inhibit or partially inhibits the activation of AT₁R. This includes antagonists for AT₁R, inverse agonists and negative allosteric modulators.

The term “AT₁R blocker” includes pharmaceutically acceptable salts of AT₁R blockers. Alternatively, the AT₁R blocker may be an antibody blocker of the AT₁R.

For example, the AT₁R blocker may be selected from the group comprising: irbesartan (e.g. Avapro®), eprosartan (e.g. Teveten®), losartan (e.g. Cozaar®), valsartan (e.g. Diovan®), telmisartan (e.g. Micardis®), candesartan (e.g. Atacand®), olmesartan (e.g. Benicar®), azilsartan (e.g. Edarbi®), sparsentan (e.g. Filspari), fimasartan (e.g. Kanarb®), and ZD-7115. As an example, the angiotensin receptor inhibitor may be irbesartan or valsartan.

Taylor et al (2011) (Journal of the American Society of Hypertension 13 (9): 677-686) reviews angiotensin II AT₁ receptor blockers (also known as AT₁R blockers or ARBs) and notes that “[t]here are no major differences in efficacy or other clinical characteristics among older drugs in this class”. It is noted that differences in characteristics appear to be related to interaction with other pathways and not the core angiotensin II AT₁ receptor blockage. Taylor reports that although “[t]here are some pharmacological differences among the ARBs, and some drugs in this class have special effects independent of angiotensin II receptor blockade, yet there is currently no strong evidence that these translate into unique protection against target organ damage from hypertension”. Thus, in respect of their effect on AT₁R blockage, ARBs are functionally interchangeable despite structural differences.

As noted in the first paragraph of Taylor, ARBs first become commercially available in the 1990's, and eight specific ARBs are cited as being available in the USA. Despite the extended period of use of this class of drugs, physicians appear to treat these as a group which are largely substitutable with each other. While there are some specific subtleties in the exact pharmacology of some of the group, these appear not to be related to their angiotensin II AT₁ receptor activity. Furthermore, all pharmacological products will behave subtly differently in some patients, and yet some sub-group variability does not prevent them from being generally useful in therapy.

The AT₁R blocker optionally inhibits or partially inhibits the AT₁R. Optionally, the AT₁R blocker directly interacts with the AT₁R, and does not include therapeutics such as angiotensin converting enzyme inhibitors (ACEi) therapeutics that act upstream of the AT₁R to prevent generation of the functional ligand but that do not interact with the AT₁R itself.

The term “AT₁R blocker” includes pharmaceutically acceptable salts of AT₁R blockers. Alternatively, the AT₁R blocker may be an antibody blocker of the AT₁R.

As an example, the angiotensin receptor inhibitor may be irbesartan. Irbesartan is an angiotensin type 1 receptor antagonist also known as 2-butyl-3-({4-[2-(2H-1,2,3,4-tetrazol-5-yl)phenyl] phenyl}methyl)-1,3-diazaspiro[4.4]non-1-en-4-one. The angiotensin receptor inhibitor may be sparsentan. Sparsentan, also known as 2-[4-[(2-butyl-4-oxo-1,3-diazaspiro[4.4]non-1-en-3-yl)methyl]-2-(ethoxymethyl)phenyl]-N-(4,5-dimethyl-1,2-oxazol-3-yl)benzenesulfonamide, is a combined selective antagonist of the angiotensin II type 1 receptor (AT₁R) and the endothelin A receptor.

CCR2-AT1R complexes have previously been identified as a target for the treatment of renal and other diseases. The present inventors have previously demonstrated consistently high inhibition of inositol phosphate signalling by the AT₁R-CCR2 heteromer with ARBs. They have also demonstrated that the CCR2-AT₁R complex results in activation of Gαi proteins as well as recruitment of β-arrestin.

However, it is unknown if ARBs maintain their potency for inhibition of Gαi protein activation or recruitment of B-arrestin when acting at the CCR2-AT₁R complex compared to AT₁R alone. This is important because AT₁R inhibition by ARBs is required for clinical effectiveness. The potency of ARBs at AT₁R only versus the potency of ARBs at co-stimulated CCR2-AT₁R complexes in a Gαi protein dissociation assay were generally found to have a good correlation.

However, surprisingly the compound EXP3174 was found to have lower than expected potency at the CCR2-AT₁R complex than at AT₁R only. Thus, the AT₁R blocker EXP3174 does not function as effectively as other AT₁R blockers in the presence of the AT₁R-CCR2 complex. This indicates that the affinity of ARBs and the CCR2-AT₁R complex is not obvious. ARBs that have lower potency at the CCR2-AT₁R complex compared to AT₁R only may have reduced clinical effectiveness.

The AT₁R blocker EXP3174 is a metabolite of losartan that has been used extensively in research and has been reported to be significantly more potent than losartan in blocking angiotensin II-induced cellular responses (Sachinidis et al (1993) EXP3174, a metabolite of losartan (MK 954, DuP 753) is more potent than losartan in blocking the angiotensin II-induced responses in vascular smooth muscle cells J Hypertens 11 (2): 155-62). EXP3174 was used in WO2012094703 as an experimental AT₁R blocker in combination with CCR2 pathway inhibitors.

The present disclosure therefore provides a formulation comprising a therapeutically effective amount of an AT₁R blocker that has an affinity for the CCR2-AT₁R complex that is the same or greater than the AT₁R blocker's affinity for AT₁R only.

For the purpose of the present disclosure same or greater affinity of an AT₁R blocker for the CCR2-AT₁R complex is considered to be AT₁R locker affinities where there is no more than 3.5-fold loss in affinity at the CCR2-AT₁R complex compared to the affinity at the AT₁R.

The present disclosure further provides a dosing regimen comprising administering a daily dose of a therapeutically effective amount of an AT₁R blocker that has an affinity for the CCR2-AT₁R complex that is the same or greater than the AT₁R blocker's affinity for AT₁R only.

Optionally, the AT₁R blocker is chosen from the list consisting of: candesartan, eprosartan, fimasartan, irbesartan, losartan, olmesartan, telmisartan, valsartan and sparsentan. In one aspect, the AT₁R blocker is not EXP3174.

If the AT₁R blocker has a lower affinity for the CCR2-AT₁R complex than for AT₁R alone, the experimental results for dosing establishment may not be translatable into the clinical setting. The therapeutic dose of an AT₁R blocker that has a lower affinity for the CCR2-AT₁R complex than for AT₁R alone will be higher than expected and established in an experimental setting using only the AT₁R alone, as in the subject body the AT₁R is provided in a CCR2-AT₁R complex. Thus, more of the AT₁R blocker with a lower affinity for the CCR2-AT₁R complex than for AT₁R alone will be required to achieve the same therapeutic effect in the body.

Optionally, the AT₁R blocker is provided at from about 2 mg to 800 mg per day, provided in one or more doses. For example, the AT₁R blocker may be provided at from about 5 mg to 320 mg per day. For example, the AT₁R blocker is irbesartan and is administered at a dose of about 75, 150 or 300 mg per day, provided in one or more doses. Alternatively, the AT₁R blocker is valsartan and is administered at a dose of about 80 mg, 160 mg or 320 mg per day, provided in one or more doses.

As discussed above, AT₁R blockers are functionally interchangeable as described in Taylor et al (2011) although doses of the various actives are different. The skilled reader would use their knowledge of the dosage ranges for each AT₁R blocker to establish a suitable dose for use in the present disclosure.

TABLE 1
Recommended daily doses of AT₁R blockers

| ARB | Daily Dose | |
|---|---|---|
| Irbesartan | 50-300 | mg |
| Valsartan | 30-320 | mg |
| Losartan | 10-100 | mg |
| Candesartan | 2-32 | mg |
| Telmisartan | 10-80 | mg |
| Olmesartan | 5-40 | mg |
| Eprosartan | 300-800 | mg |
| Azilisartan | 10-80 | mg |
| Fimasartan | 30-120 | mg |

### Combination Formulations

Without being held to any theory, it is proposed that inhibition of CCR2 pathways as well as AT₁R signalling is preferred for clinical effectiveness. A combination of a CCR2 pathway inhibitor and an ARB that has an affinity for the CCR2-AT₁R complex that is the same or greater than AT₁R only is therefore preferred.

Examples of such combinations include repagermanium or propagermanium in combination with the ARBs candesartan, eprosartan, fimasartan, irbesartan, losartan, olmesartan, telmisartan, and valsartan as well as the dual ARB endothelin receptor antagonist sparsentan. The combinations do not include repagermanium or propagermanium in combination with EXP3174.

The disclosure further provides a formulation adapted to deliver:
a) a therapeutically effective amount of repagermanium or propagermanium; and
b) a therapeutically effective amount of an AT₁R blocker that has an affinity for the CCR2-AT₁R complex that is the same or greater than the AT₁R blocker's affinity for AT₁R only.

The disclosure further provides a formulation adapted to deliver:
a) a daily dose of from about 200 mg to 280 mg repagermanium or propagermanium; and
b) a therapeutically effective amount of an AT₁R blocker that has an affinity for the CCR2-AT₁R complex that is the same or greater than the AT₁R blocker's affinity for AT₁R only.

The present disclosure further provides a dosing regimen comprising administering a daily dose of:
a) from about 200 mg to 280 mg repagermanium or propagermanium;
b) a therapeutically effective amount of an AT₁R blocker that has an affinity for the CCR2-AT₁R complex that is the same or greater than the AT₁R blocker's affinity for AT₁R only.

The present disclosure further provides a dosing regimen comprising administering a daily dose of:
c) from about 200 mg to 280 mg repagermanium or propagermanium;
d) a therapeutically effective amount of an AT₁R blocker that has an affinity for the CCR2-AT₁R complex that is the same or greater than the AT₁R blocker's affinity for AT₁R only.

The AT₁R blocker that has an affinity for the CCR2-AT₁R complex that is the same or greater than the AT₁R blocker's affinity for AT₁R only may be chosen from the list consisting of: candesartan, eprosartan, fimasartan, irbesartan, losartan, olmesartan, telmisartan, valsartan and sparsentan. In one aspect, the AT₁R blocker is not EXP3174.

For the purpose of the present disclosure same or greater affinity of an AT₁R blocker for the CCR2-AT₁R complex is considered to be AT₁R blocker affinities where there is no more than 3.5-fold loss in affinity at the CCR2-AT₁R complex compared to the affinity at the AT₁R.

The therapeutically effective amount of AT₁R blocker above may be a daily dose of from about 2 mg to 800 mg.

The pharmaceutical formulations described above may optionally include excipients, solvents, carriers and other pharmaceutically acceptable ingredients.

The term “component” as used herein in the context of a pharmaceutical formulation of the disclosure, means either the AT₁R blocker or repagermanium.

The term “inhibits” as used herein, means a reduction below detectable limits when compared to a reference. The phrase includes blocking, retarding, or impeding an action to prevent an undesirable result.

The term “partially inhibits” as used herein, means any reduction within detectable limits when compared to a reference. The phrase includes blocking, retarding, or impeding an action to prevent an undesirable result.

Both the repagermanium and the AT₁R blocker may be the same active agent, for example a bi-specific antibody.

Both the AT₁R blocker and the repagermanium may be pharmaceutically acceptable salts of the respective active agent. Pharmaceutically and veterinary acceptable salts include salts which retain the biological effectiveness and properties of the compounds of the present disclosure and which are not biologically or otherwise undesirable. In many cases, the compounds disclosed herein are capable of forming acid and/or base salts by virtue of the presence of amino and/or carboxyl groups or groups similar thereto. Acceptable base addition salts can be prepared from inorganic and organic bases. Salts derived from inorganic bases, include by way of example only, sodium, potassium, lithium, ammonium, calcium and magnesium salts. Salts derived from organic bases include, but are not limited to, salts of primary, secondary and tertiary amines, such as by way of example only, alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amines, trisubstituted cycloalkyl amines, cycloalkenyl amines, di(cycloalkenyl) amines, tri(cycloalkenyl) amines, substituted cycloalkenyl amines, disubstituted cycloalkenyl amines, trisubstituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and tri-amines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heteroaryl, heterocyclic, and the like. Also included are amines where the two or three substituents, together with the amino nitrogen, form a heterocyclic or heteroaryl group.

Pharmaceutically and veterinary acceptable acid addition salts may be prepared from inorganic and organic acids. The inorganic acids that can be used include, by way of example only, hydrochloric acid, hydrobromic acid, sulfuric acid, nitric acid, phosphoric acid, and the like. The organic acids that can be used include, by way of example only, acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

The pharmaceutically or veterinary acceptable salts of the compounds useful in the present disclosure can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile may be used. Lists of suitable salts are found in Remington's Pharmaceutical Sciences. 17^{th} ed., Mack Publishing Company, Easton, Pa. (1985), p. 1418, the disclosure of which is hereby incorporated by reference. Examples of such acceptable salts are the iodide, acetate, phenyl acetate, trifluoroacetate, acryl ate, ascorbate, benzoate, chlorobenzoate, dinitrobenzoate, hydroxybenzoate, methoxybenzoate, methylbenzoate, o-acetoxybenzoate, naphthalene-2-benzoate, bromide, isobutyrate, phenylbutyrate, y-hydroxybutyrate, β-hydroxybutyrate, butyne-1,4-dioate, hexyne-1,4-dioate, hexyne-1,6-dioate, caproate, caprylate, chloride, cinnamate, citrate, decanoate, formate, fumarate, glycollate, heptanoate, hippurate, lactate, malate, maleate, hydroxymaleate, malonate, mandelate, mesylate, nicotinate, isonicotinate, nitrate, oxalate, phthalate, terephthalate, phosphate, monohydrogenphosphate, dihydrogenphosphate, metaphosphate, pyrophosphate, propiolate, propionate, phenylpropionate, salicylate, sebacate, succinate, suberate, sulfate, bisulfate, pyrosulfate, sulfite, bisulfite, sulfonate, benzenesulfonate, p-bromophenylsulfonate, chlorobenzenesulfonate, propanesulfonate, ethanesulfonate, 2-hydroxyethanesulfonate, merhanesulfonate, naphthalene-I-sulfonate, naphthalene-2-sulfonate, p-toluenesulfonate, xylenesulfonate, tartarate, and the like.

### Condition or Disease

Generally, a range of ailments which are chemokine-related may be treated by the method of the present disclosure. This includes ailments that are related to increased or decreased production of chemokines, and/or increased or decreased responsiveness of cells to chemokines. A chemokine-related ailment should also be understood to mean a disease or condition in which chemokine receptors display aberrant characteristics, are the target of a particular pathogen or are a target of a pharmacological intervention.

The ailments which are chemokine-related may be inflammatory conditions, or conditions deriving from or associate with inflammation. The inflammatory condition or condition deriving from or associate with inflammation may be a kidney disease.

The inflammatory condition or condition deriving from or associate with inflammation to be treated, ameliorated or prevented may be a kidney disease. For example, the kidney disease is a kidney disease associated with proteinuria. The kidney disease may be chosen from the list comprising: focal segmental glomerulosclerosis (FSGS; including idiopathic (primary) FSGS, secondary FSGS, genetic FSGS, and any other causes of FSGS), fibrotic disorders in the kidney, Immunoglobulin A nephropathy (IgAN), Alport's Syndrome, chronic kidney disease including chronic kidney disease caused by diabetic nephropathy, renal insufficiency (diabetic and non-diabetic), and renal failure conditions, including diabetic nephropathy, glomerulonephritis, scleroderma, glomerular sclerosis, proteinuria of primary renal disease and renal vascular hypertension.

The term “prevention” includes administration of the formulations of the present disclosure to subjects who have symptoms that indicate they are at risk of developing a kidney disease as indicated above, but may not, at the time of treatment, be diagnosed with the kidney disease. For example, the subject may have elevated blood creatine or urinary protein, but not yet be diagnosed with kidney disease.

The term “amelioration” includes administration of the formulations of the present disclosure to subjects who have had a kidney disease and are at risk of developing post-disease conditions.

### Measurement of Inhibition

The inhibition or partial inhibition of the CCR2 pathway and/or the AT₁R caused by (i) the repagermanium or propagermanium, (ii) the AT₁R blocker, or (iii) a combination of both the repagermanium or propagermanium and the AT₁R blocker, may be measured using the in vitro methods set out herein, and include but are not limited to, biochemical or cellular assays for the assessment of in vitro chemotactic migration of CCR2-expressing neutrophils and other cells such as are known in the art, as well as measurement of inositol phosphate production, extracellular-regulated kinase (ERK) phosphorylation, cAMP production, label-free technologies (such as using impedance, light refraction or charge redistribution), G protein coupling using proximity reporter systems or other approaches, β-arrestin recruitment or mediated signalling, transcription factor-based reporter systems, microscopy visualization using fluorescent labels, use of antibodies to assess receptor cellular localization (such as enzyme-linked immunosorbent assays), use of bioluminescence resonance energy transfer to assess cellular localization and trafficking, and fluorescence activated cell sorting.

The inhibition or partial inhibition of the CCR2 pathway and/or the AT₁R caused by (i) the repagermanium or propagermanium, (ii) the AT₁R blocker, or (iii) a combination of both the repagermanium or propagermanium and the AT₁R blocker, may be measured using the in vivo methods including but not limited to, measurement of cellular and cytokine content of lung exudate, measurement of lung function including physical capacity of lung function using spirometry-based tests, or lung functional outputs measured using measurement blood gas or other biochemical measures, or improvement in functional benefit including clinical benefit measured by quantitative methods such as walk tests or qualitative methods such as patient-reported outcome assessment. Inhibition or partial inhibition may be indicated by a qualitative improvement in lung structure as measured by one or more of the above-mentioned endpoints.

In one embodiment, the total efficacy of the pharmaceutical formulation is greater when compared to the efficacies of the AT₁R blocker or the repagermanium or propagermanium when either component is administered without any administration of the other component. Thus, the combined formulation may be administered in a single dose, including at sub-therapeutic doses, or less often, than either of the two components might be administered as single compounds.

Optionally, the total efficacy of the pharmaceutical formulation is greater when compared to the sum of the efficacies of the AT₁R blocker and the repagermanium or propagermanium when either component is administered without any administration of the other component. A synergistic effect in efficacy may be observed when the AT₁R blocker and the repagermanium or propagermanium are administered concurrently or sequentially.

Alternatively, the total efficacy of the pharmaceutical formulation may be equal to the sum of the efficacies of the AT₁R blocker and the repagermanium or propagermanium when either component is administered without any administration of the other component. As a further embodiment of this alternative, an additive effect in efficacy is observed when the AT₁R blocker and the repagermanium or propagermanium are administered concurrently or sequentially.

In a further alternative, the total efficacy of the pharmaceutical formulation is less than the sum of the efficacies of the AT₁R blocker and the repagermanium or propagermanium when either component is administered without any administration of the other component. In a further embodiment, while the combined efficacy is less than the sum of the efficacies of the AT₁R blocker and the repagermanium or propagermanium when each component is administered without any administration of the other component, the treatment provides greater efficacy compared to a single treatment of AT₁R blocker or the repagermanium or propagermanium administered alone.

Optionally, the two components are administered concurrently at the same time (for example as two tablets taken together, or as a single tablet, formulated with each component) or sequentially (for example one tablet taken after another tablet). The doses of each component may be taken together (concurrently), or sequentially and taken within seconds, minutes, days, weeks or months of each other.

One component of the combination of the present disclosure may already be being administered to a subject, for example as standard of care treatment. In such a case, the second component of the combination of the present disclosure is administered as a second component in therapy to provide the therapeutic combination of the present disclosure.

### Method of Treatment

The disclosure further provides a method for treatment, amelioration or prevention of a kidney disease, said method comprising the step of administering to a subject:
a) a therapeutic formulation adapted to deliver from about 200 mg to 280 mg repagermanium or propagermanium per day.

The disclosure further provides a method for treatment, amelioration or prevention of a kidney disease, said method comprising the step of administering to a subject:
i) a therapeutically effective amount of an AT₁R blocker that has an affinity for the CCR2-AT₁R complex that is the same or greater than the AT₁R blocker's affinity for AT₁R only.

The disclosure further provides a method for treatment, amelioration or prevention of a kidney disease, said method comprising the step of administering to a subject:
i) a therapeutically effective amount of repagermanium or propagermanium per day; and
ii) a therapeutically effective amount of an AT₁R blocker that has an affinity for the CCR2-AT₁R complex that is the same or greater than the AT₁R blocker's affinity for AT₁R only.

The disclosure further provides a method for treatment, amelioration or prevention of a kidney disease, said method comprising the step of administering to a subject:
i) a therapeutic formulation adapted to deliver from about 200 mg to 280 mg repagermanium or propagermanium per day; and
ii) a therapeutically effective amount of an AT₁R blocker that has an affinity for the CCR2-AT₁R complex that is the same or greater than the AT₁R blocker's affinity for AT₁R only.

The subject to be treated may be a mammal, including a human mammal.

The repagermanium and the AT₁R blocker may be administered: in the same dosage form or in separate dosage forms.

The repagermanium and/or the AT₁R blocker may be antibody inhibitors or blockers of the respective receptors. The repagermanium and the AT₁R blocker may be the same active agent, for example a bi-specific antibody. The repagermanium and/or the AT₁R blocker may be pharmaceutically acceptable salts of the repagermanium and/or the AT₁R blocker.

The repagermanium and the AT₁R blocker may be administered: concurrently or sequentially.

One component of the combination of the present disclosure may already be being administered to a subject, for example as standard of care treatment. In such a case, the second component of the combination of the present disclosure is administered as a second component in therapy to provide the therapeutic combination of the present disclosure.

While not intending to be restricted to any particular mode of action, in one embodiment the repagermanium has a greater affinity and/or potency and/or efficacy when interacting with the CCR2 or modulating its downstream pathways when the CCR2 is associated with the angiotensin receptor AT₁R. For example, the CCR2 and the angiotensin receptor AT₁R may be associated as a CCR2/AT₁R complex.

Optionally, the combination of the present disclosure provides a lower dose of the AT₁R blocker or repagermanium than when either one is used alone. For example, one or both of the AT₁R blocker or repagermanium may be provided at a subtherapeutic dose. This would have the benefit of reducing the negative safety profile of the AT₁R or repagermanium while having the same therapeutic benefit.

In a further embodiment, when the repagermanium is administered to a subject concurrently or sequentially with an AT₁R blocker, the combined affinity, potency and/or efficacy is greater than compared to the affinity, potency and/or efficacy that would have been achieved when the repagermanium is not administered in combination (whether concurrently or sequentially) with the AT₁R blocker. In an even further embodiment, a synergistic effect (as measured by affinity, potency and/or efficacy) is achieved when the repagermanium is administered to a subject in combination (whether concurrently or sequentially) with an AT₁R blocker.

While not intending to be restricted to any particular mode of action, in one embodiment the AT₁R blocker has a greater affinity and/or potency and/or efficacy when interacting with the angiotensin receptor AT₁R when the angiotensin receptor AT₁R is associated with the CCR2. For example, the CCR2 and the angiotensin receptor AT₁R may be associated as a CCR2/AT₁R complex. In a further embodiment, when the AT₁R blocker is administered to a subject concurrently or sequentially with a repagermanium, the combined affinity, potency and/or efficacy is greater than compared to the affinity, potency and/or efficacy that would have been achieved when the AT₁R blocker is not administered in combination (whether concurrently or sequentially) with the repagermanium. In a further embodiment, a synergistic effect (as measured by affinity, potency and/or efficacy) is achieved when the AT₁R blocker is administered to a subject in combination (whether concurrently or sequentially) with a repagermanium.

For the purpose of the present disclosure same or greater affinity of an AT₁R blocker for the CCR2-AT₁R complex is considered to be AT₁R locker affinities where there is no more than 3.5-fold loss in affinity at the CCR2-AT₁R complex compared to the affinity at the AT₁R.

### Delivery

The dosage form provided by the present disclosure may further comprise a vial, cartridge, container, tablet or capsule comprising the pharmaceutical formulation of the disclosure together with dosage instructions for the administration of the dosage form to a subject for the treatment, amelioration or prevention of a condition or disease.

The amount of each active ingredient which may be combined with the carrier materials to produce a single dosage will vary, depending upon the host to be treated and the particular mode of administration. For example, a formulation intended for oral administration to humans may contain about 0.5 mg to 1 g of each active compound with an appropriate and convenient amount of carrier material, which may vary from about 5 to 95 percent of the total formulation. Dosage unit forms will generally contain between from about 0.5 mg to 500 mg of active ingredient(s).

Optionally, the AT₁R blocker is provided at from about 2 mg to 800 mg per day, provided in one or more doses. For example, the AT₁R blocker may be provided at from about 75 mg to 300 mg per day. For example, the AT₁R blocker is irbesartan and is administered at a dose of about 75, 150 or 300 mg per day, provided in one or more doses.

Optionally, the repagermanium is provided at from about 200 mg to 280 mg repagermanium or propagermanium per day, provided in one or more doses. For example, the repagermanium or propagermanium is provided at a dose of from about 220 mg to 260 mg per day, or about 240 mg per day, provided in one or more doses.

The dose of each active agent may be provided in either a single dosage form, or two separate dosage forms and may comprise from about 2 mg to 800 mg of the AT₁R blocker, and from about 200 mg to 280 mg of the repagermanium or propagermanium. The dose of the two actives may be provided in either a single dosage form, or two separate dosage forms and may comprise (i) a daily dose of AT₁R blocker of from about 2 mg to 800 mg, and (ii) a daily dose of repagermanium or propagermanium of from about 200 mg to 280 mg. The AT₁R blocker may be irbesartan, and the dosage form may comprise a daily dose of irbesartan of about 300 mg. Alternatively, the AT₁R blocker is valsartan and is administered at a dose of about 80 mg, 160 mg or 320 mg per day, provided in one or more doses.

It will be understood, however, that the specific dose level for any particular subject will depend upon a variety of factors including the activity of the specific compound employed, the age, body weight, general health, sex, diet, time of administration, route of administration, rate of excretion, drug combination and the severity of the particular condition or disease undergoing therapy.

Formulations of the disclosure, in various aspects, may be administered by injection, or prepared for oral, pulmonary, nasal or for any other form of administration. The formulations may be administered, for example, intravenously, subcutaneously, intramuscularly, intraorbitally, ophthalmically, intraventricularly, intracranially, intracapsularly, intraspinally, intracisternally, intraperitoneally, buccal, rectally, vaginally, intranasally or by aerosol administration.

The mode of administration is in one aspect at least suitable for the form in which the formulation has been prepared. The mode of administration for the most effective response may be determined empirically and the means of administration described below are given as examples, and do not limit the method of delivery of the formulation of the present disclosure in any way. All the formulations provided are commonly used in the pharmaceutical industry and are commonly known to suitably qualified practitioners.

### Oral Dosage Forms

Contemplated for use herein are oral solid dosage forms, which are described generally in Martin, Remington's Pharmaceutical Sciences, 18th Ed. (1990 Mack Publishing Co. Easton PA 18042) at Chapter 89, which is herein incorporated by reference. Solid dosage forms include tablets, capsules, pills, troches or lozenges, cachets or pellets. Also, liposomal or proteinoid encapsulation may be used to formulate the present formulations (as, for example, proteinoid microspheres reported in U.S. Pat. No. 4,925,673). Liposomal encapsulation may be used and the liposomes may be derivatised with various polymers (E.g., U.S. Pat. No. 5,013,556). A description of possible solid dosage forms for the therapeutic is given by Marshall, in Modern Pharmaceutics, Chapter 10, Banker and Rhodes ed., (1979), herein incorporated by reference. In general, the formulation will include the compounds described as part of the disclosure (or a chemically modified form thereof), and inert ingredients which allow for protection against the stomach environment, and release of the biologically active material in the intestine.

The oral dosage form of the present disclosure may be a liquid, suspension or other appropriate dosage form that is delivered directly into the feeding tube of a patient who is in a coma and/or on a respirator and is unable to swallow a tablet etc.

For the repagermanium or AT₁R blocker of the disclosure the location of release may be the stomach, the small intestine (the duodenum, the jejunum, or the ileum), or the large intestine. One skilled in the art has available formulations that will not dissolve in the stomach yet will release the material in the duodenum or elsewhere in the intestine. In one aspect, the release will avoid the deleterious effects of the stomach environment, either by protection of the formulation or by release of the compounds beyond the stomach environment, such as in the intestine.

A coating or mixture of coatings can also be used on tablets, which are not intended for protection against the stomach. This includes without limitation sugar coatings, or coatings that make the tablet easier to swallow. Exemplary capsules consist of a hard shell (such as gelatine) for delivery of dry therapeutic i.e. powder; for liquid forms, a soft gelatine shell may be used. The shell material of cachets in certain aspects is thick starch or other edible paper. For pills, lozenges, moulded tablets or tablet triturates, moist massing techniques are also contemplated, without limitation.

The disclosure further provides an oral sustained release pharmaceutical formulation comprising a therapeutically effective pharmaceutical formulation according to the disclosure, and a release retardant.

In one aspect of the present disclosure the release retardant is a water-soluble, water swellable and/or water insoluble polymer. In particular, water-soluble polymers are selected from the group comprising ethylcellulose, hydroxypropyl cellulose, hydroxypropyl methyl cellulose, an enteric coating; and a semipermeable membrane. In another aspect of the disclosure the release retardant is a non-polymeric release retardant. More particularly, the non-polymeric release retardant is hydrogenated castor oil. The formulations of the disclosure may be milled or granulated and compressed into tablets or encapsulated into capsules according to conventional procedures known in the art.

To ensure full gastric resistance, a coating impermeable to at least pH 5.0 is used. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

As used herein, the term “sustained release” means the gradual but continuous or sustained release over a relatively extended period of the therapeutic compound content after oral ingestion. The release may continue after the pharmaceutical formulation has passed from the stomach and through until and after the pharmaceutical formulation reaches the intestine. The phrase “sustained release” also means delayed release wherein release of the therapeutic compound is not immediately initiated upon the pharmaceutical formulation reaching the stomach but rather is delayed for a period of time, for example, until when the pharmaceutical formulation reaches the intestine. Upon reaching the intestine, the increase in pH may then trigger release of the therapeutic compound from the pharmaceutical formulation.

The term “release retardant” as used herein means a substance that reduces the rate of release of a therapeutic compound from a pharmaceutical formulation when orally ingested. The release retardant may be a polymer or a non-polymer. The release retardant may be used according to any one of several sustained release systems including, for example, a diffusion system, a dissolution system and/or an osmotic system.

In certain aspects, the therapeutic is included in the formulation as fine multiparticulates in the form of granules or pellets of particle size about 1 mm. The formulation of the material for capsule administration is, in certain aspects, a powder, lightly compressed plugs or even as tablets. In one aspect, the therapeutic could be prepared by compression.

Colourants and flavouring agents may optionally be included. For example, compounds may be formulated (such as, and without limitation, by liposome or microsphere encapsulation) and then further contained within an edible product, such as a refrigerated beverage containing colorants and flavouring agents.

The volume of the therapeutics may, in one aspect, be diluted or increased with an inert material. These diluents could include carbohydrates, especially mannitol, alpha-lactose, anhydrous lactose, cellulose, sucrose, modified dextrans and starch. Certain inorganic salts are also optionally used as fillers including calcium triphosphate, magnesium carbonate and sodium chloride. Some commercially available diluents are Fast-Flo, Emdex, STA-Rx 1500, Emcompress and Avicell.

In other embodiments, disintegrants are included in the formulation of the therapeutic into a solid dosage form. Materials used as disintegrants include but are not limited to starch including the commercial disintegrant based on starch, Explotab. Sodium starch glycolate, Amberlite, sodium carboxymethylcellulose, ultramylopectin, sodium alginate, gelatine, orange peel, acid carboxymethyl cellulose, natural sponge and bentonite are also contemplated. Another form of the disintegrants is the insoluble cationic exchange resins. Powdered gums are also optionally used as disintegrants and as binders and these include, without limitation, powdered gums such as agar, Karaya or tragacanth. Alginic acid and its sodium salt are also useful as disintegrants.

Binders are contemplated to hold the therapeutic compounds together to form a hard tablet and include, without limitation, materials from natural products such as acacia, tragacanth, starch and gelatine. Other binders include, without limitation, methylcellulose (MC), ethyl cellulose (EC) and carboxymethyl cellulose (CMC). Polyvinyl pyrrolidone (PVP) and hydroxypropylmethyl cellulose (HPMC) are contemplated for use in alcoholic solutions to granulate the therapeutic.

An antifrictional agent may be optionally included in the formulation of the therapeutic to prevent sticking during the formulation process. Lubricants may be optionally used as a layer between the therapeutic and the die wall, and these can include but are not limited to: stearic acid including its magnesium and calcium salts, polytetrafluoroethylene (PTFE), liquid paraffin, vegetable oils and waxes. Exemplary soluble lubricants may also be used such as include sodium lauryl sulfate, magnesium lauryl sulfate, polyethylene glycol of various molecular weights, and Carbowax 4000 and 6000.

Glidants that might improve the flow properties of the compound during formulation and to aid rearrangement during compression might be optionally added. The glidants may include without limitation starch, talc, pyrogenic silica and hydrated silicoaluminate.

To aid dissolution of the therapeutic into the aqueous environment, a surfactant might be added in certain embodiments as a wetting agent. Surfactants may include, for example and without limitation, anionic detergents such as sodium lauryl sulfate, dioctyl sodium sulfosuccinate and dioctyl sodium sulfonate. Cationic detergents might be optionally used and could include, without limitation, benzalkonium chloride or benzethomium chloride. The list of potential nonionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. When used, these surfactants could be present in the formulation of the compounds either alone or as a mixture in different ratios.

Additives that potentially enhance uptake of the compounds may be desirable. Such additives include the fatty acids oleic acid, linoleic acid and linolenic acid.

Controlled release formulation may be desirable. In certain aspects, the compounds could be incorporated into an inert matrix that permits release by either diffusion or leaching mechanisms i.e., gums. In some aspects, slowly degenerating matrices may also be incorporated into the formulation. Another form of a controlled release of this therapeutic is by a method based on the OROS™ therapeutic system (Alza Corp.), i.e. the drug is enclosed in a semipermeable membrane which allows water to enter and push drug out through a single small opening due to osmotic effects. Some enteric coatings also have a delayed release effect.

In other aspects, a mix of materials might be used to provide the optimum film coating. Film coating may be carried out, for example and without limitation, in a pan coater or in a fluidized bed or by compression coating.

### Dosing Schedule

It will be appreciated that in certain aspects, the formulations of the disclosure may be given as a single dose schedule, or in a multiple dose schedule. A multiple dose schedule is one in which a primary course of delivery may be with 1 to 10 separate doses, is optionally followed by other doses given at subsequent time intervals required to maintain or reinforce the treatment. The dosage regimen will also, at least in part, be determined by the needs of the individual and the judgement of the practitioner.

The disclosure thus provides a tablet, capsule, liquid, suspension or other oral dosage form comprising the pharmaceutical formulation of the disclosure.

The AT₁R blocker and the repagermanium or propagermanium may be delivered in the same formulation, or may be delivered in separate formulations.

The AT₁R blocker and the repagermanium or propagermanium may be in the same dosage form, or may be in separate dosage forms.

The subject being administered the AT₁R blocker and the repagermanium may be already receiving one of the active agents and may, in accordance with the present disclosure, be administered the other component of the treatment of the present disclosure.

### Use

The present disclosure provides for the use of a daily dose of from about 200 mg to 280 mg repagermanium or propagermanium for the manufacture of a formulation for the treatment, amelioration or prevention of a kidney disease.

The present disclosure provides for the use of a daily dose of from about 200 mg to 280 mg repagermanium or propagermanium for the treatment, amelioration or prevention of a kidney disease.

The present disclosure provides for the use of a therapeutically effective amount of an AT₁R blocker that has an affinity for the CCR2-AT₁R complex that is the same or greater than the AT₁R blocker's affinity for AT₁R only for the manufacture of a formulation for the treatment, amelioration or prevention of a kidney disease.

The present disclosure provides for the use of a therapeutically effective amount of an AT₁R blocker that has an affinity for the CCR2-AT₁R complex that is the same or greater than the AT₁R blocker's affinity for AT₁R only for the treatment, amelioration or prevention of a kidney disease.

The disclosure also provides for the use of (a) a therapeutically effective amount of repagermanium or propagermanium, and (b) a therapeutically effective amount of an AT₁R blocker that has an affinity for the CCR2-AT₁R complex that is the same or greater than the AT₁R blocker's affinity for AT₁R only for the manufacture of a formulation for the treatment, amelioration or prevention of a kidney disease.

The disclosure also provides for the use of (a) a therapeutically effective amount of repagermanium or propagermanium, and (b) a therapeutically effective amount of an AT₁R blocker that has an affinity for the CCR2-AT₁R complex that is the same or greater than the AT₁R blocker's affinity for AT₁R only for the treatment, amelioration or prevention of a kidney disease.

The present disclosure further provides a therapeutically effective amount of repagermanium or propagermanium for use in a formulation for the treatment, amelioration or prevention of a kidney disease wherein the at least one AT₁R blocker is administered to the subject concurrently or sequentially with repagermanium or propagermanium.

The present disclosure further provides a therapeutically effective amount of repagermanium or propagermanium for use in a formulation for the treatment, amelioration or prevention of a kidney disease wherein the repagermanium is administered to the subject concurrently or sequentially with a therapeutically effective amount of an AT₁R blocker that has an affinity for the CCR2-AT₁R complex that is the same or greater than the AT₁R blocker's affinity for AT₁R only.

The therapeutically effective amount of repagermanium or propagermanium above may be a daily dose of from about 200 mg to 280 mg repagermanium or propagermanium.

The AT₁R blocker that has an affinity for the CCR2-AT₁R complex that is the same or greater than the AT₁R blocker's affinity for AT₁R only may be chosen from the list consisting of: candesartan, eprosartan, fimasartan, irbesartan, losartan, olmesartan, telmisartan, valsartan and sparsentan. In one embodiment, the AT₁R blocker is not EXP3174.

The therapeutically effective amount of AT₁R blocker above may be a daily dose of from about 2 mg to 800 mg.

The repagermanium or propagermanium and the AT₁R blocker may be administered in the same dosage form or in separate dosage forms.

The repagermanium or propagermanium and/or the AT₁R blocker may be an antibody inhibitor or blocker of the respective receptors. The repagermanium or propagermanium and the AT₁R blocker may be the same active agent, for example a bi-specific antibody. The repagermanium or propagermanium and/or the AT₁R blocker may be pharmaceutically acceptable salts of the repagermanium or propagermanium and/or the AT₁R blocker.

The repagermanium or propagermanium and the AT₁R blocker may be administered: concurrently or sequentially.

### Kits

The present disclosure provides a kit for the treatment or prevention of a kidney disease, said kit comprising:
a) a daily dose of from about 200 mg to 280 mg repagermanium or propagermanium; and
b) instructions for use.

The disclosure further provides a kit for the treatment or prevention of a kidney disease, said kit comprising:
a) a therapeutically effective amount of an AT₁R blocker that has an affinity for the CCR2-AT₁R complex that is the same or greater than the AT₁R blocker's affinity for AT₁R only;
b) instructions for use.

The present disclosure provides a kit for the treatment or prevention of a kidney disease, said kit comprising:
a) a therapeutically effective amount of repagermanium or propagermanium;
b) a therapeutically effective amount of an AT₁R blocker that has an affinity for the CCR2-AT₁R complex that is the same or greater than the AT₁R blocker's affinity for AT₁R only; and
c) instructions for use.

The therapeutically effective amount of repagermanium or propagermanium above may be a daily dose of from about 200 mg to 280 mg repagermanium or propagermanium.

The AT₁R blocker that has an affinity for the CCR2-AT₁R complex that is the same or greater than the AT₁R blocker's affinity for AT₁R only may be chosen from the list consisting of: candesartan, eprosartan, fimasartan, irbesartan, losartan, olmesartan, telmisartan, valsartan and sparsentan. In one embodiment, the AT₁R blocker is not EXP3174.

The contents of the kit can be lyophilized and the kit can additionally contain a suitable solvent for reconstitution of the lyophilized components. Individual components of the kit would be packaged in separate containers and, associated with such containers, can be a notice in the form prescribed by a governmental agency regulating the manufacture, use or sale of pharmaceuticals or biological products, which notice reflects approval by the agency of manufacture, use or sale for human administration.

When the components of the kit are provided in one or more liquid solutions, the liquid solution can be an aqueous solution, for example a sterile aqueous solution. For in vivo use, the expression construct may be formulated into a pharmaceutically acceptable syringeable formulation. In this case the container means may itself be an inhalant, syringe, pipette, eye dropper, or other such like apparatus, from which the formulation may be applied to an affected area of the animal, such as the lungs, injected into an animal, or even applied to and mixed with the other components of the kit.

The components of the kit may also be provided in dried or lyophilized forms. When reagents or components are provided as a dried form, reconstitution generally is by the addition of a suitable solvent. It is envisioned that the solvent also may be provided in another container means. Irrespective of the number or type of containers, the kits of the disclosure also may comprise, or be packaged with, an instrument for assisting with the injection/administration or placement of the ultimate complex formulation within the body of an animal. Such an instrument may be an inhalant, syringe, pipette, forceps, measured spoon, eye dropper or any such medically approved delivery vehicle.

### General

Those skilled in the art will appreciate that the invention described herein is susceptible to variations and modifications other than those specifically described. The invention includes all such variation and modifications. The invention also includes all of the steps, features, formulations and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

Any manufacturer's instructions, descriptions, product specifications, and product sheets for any products mentioned herein or in any document incorporated by reference herein, are hereby incorporated herein by reference, and may be employed in the practice of the invention.

The present invention is not to be limited in scope by any of the specific embodiments described herein. These embodiments are intended for the purpose of exemplification only. Functionally equivalent products, formulations and methods are clearly within the scope of the invention as described herein.

The invention described herein may include one or more range of values (eg. Size, displacement and field strength etc). A range of values will be understood to include all values within the range, including the values defining the range, and values adjacent to the range which lead to the same or substantially the same outcome as the values immediately adjacent to that value which defines the boundary to the range. Accordingly, unless indicated to the contrary, the numerical parameters set forth in the specification and claims are approximations that may vary depending upon the desired properties sought to be obtained by the present invention. Hence “about 80%” means “about 80%” and also “80%”. At the very least, each numerical parameter should be construed in light of the number of significant digits and ordinary rounding approaches.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs. The term “active agent” may mean one active agent, or may encompass two or more active agents.

The following examples serve to more fully describe the manner of using the above-described invention, as well as to set forth the best modes contemplated for carrying out various aspects of the invention. It is understood that these methods in no way serve to limit the true scope of this invention, but rather are presented for illustrative purposes.

### EXAMPLES

Further features of the present invention are more fully described in the following non-limiting Examples. This description is included solely for the purposes of exemplifying the present invention. It should not be understood as a restriction on the broad description of the invention as set out above.

### Example 1

### Pharmacokinetic Model of Repagermanium

### Methods

Pharmacokinetic data were available from studies DMX-200-101, DMX-200-201, DMX-200-202 and DMX-200-203.

TABLE 2
Pharmacokinetic data

| Item | Study 101 | Study 202 | Study 203 |
|---|---|---|---|
| Study no. | DMX-200-101 Phase I | DMX-200-202 Phase II | DMX-200-203 Phase II |
| Phase | | | |
| Subjects | 13 | 8 | 45 |
| with PK | | | |
| Description/ | Open-label, two-way | A Double-blind, | A Phase 2, Double-blind, |
| Patient | crossover/healthy | Randomised, Placebo- | Randomised, Placebo- |
| Population | volunteers | Controlled, 2-way crossover | Controlled, Crossover Study |
| | | Study evaluating the Safety | Evaluating the Safety and |
| | | and Efficacy of RPG in | Efficacy of RPG in patients |
| | | Patients with FSGS who are | with DKD who are receiving |
| | | receiving Irbesartan | Irbesartan |
| Route | Oral, single-dose | Two treatment periods of 16 | Two treatment periods of 12 |
| Dosage/ | administrations of: | weeks | weeks |
| Formulation | IR formulation without | Oral, repeat-dose of 120 mg | Oral, repeat-dose of 120 mg |
| | food1) | RPG bid IR in period 1 and | RPG bid IR in period 1 and |
| | ER formulation without | placebo in period 2 or cross- | placebo in period 2 or cross- |
| | food | over oral, repeat-dose of | over oral, repeat-dose of |
| | ER formulation with food | placebo in period 1 and 120 | placebo in period 1 and 120 |
| | | mg RPG bid IR in period 2 | mg RPG bid IR in period 2 |
| No of | ±16 PK plasma samples | Pre-dose, week 1, 8, 15 and | Pre-dose, week 1, 6, 11 and |
| samples | per subject per treatment; 7 | 16 at both periods; 24 h | 12 at both periods; 24 h |
| | intervals of urine sampling | urine collections at ± week | urine collections at ± week |
| | | 8, 15 and 16 samples | 8, 15 and 16 samples |

In all studies blood samples were analyzed using liquid chromatography tandem mass spectrometry (LC-MS/MS). This method involves the addition of deuterated-PPG (d₂-PPG) as an internal standard (IS) and liquid-liquid extraction with chloroform for sample processing. The sample is analyzed on an LC-MS/MS system consisting of a Shimadzu LC20AD with Thermo Fisher Hypercarb column using gradient elution and an API 4000 mass spectrometer. The mass spectrometer is operated in the negative electrospray ionization (ESI) ion mode, and used to monitor the precursor to product ion transitions for PPG and d₂-PPG, respectively. The lower limit of quantification (LLOQ) was 5 ng/ml.

The pharmacometric analysis was carried out in several steps driven by the suitability of the clinical data for development of the PK model. 1. An initial structural population PK model was developed using data from healthy volunteers (study 101). 2. This model was subsequently updated using the sparse data in patients from studies 202 and 203, and effects of covariates were explored. 3. Model qualification was performed. 4. Finally, the developed model was used for simulations of the influence of covariates on key PK parameters as well as on potential exposures.

The SAS software (version 9.4) was used for preparing the analysis dataset. The population PK analysis was performed using NONMEM (version 7.5.0) and the first order conditional estimation (FOCE) method with interaction. PsN (version≥5.0.0) was used to execute NONMEM runs as well as for running visual predictive checks (VPCs), sampling importance resampling (SIR) and automated step-wise covariate modelling (SCM). The R software (version≥4.0.2) was used for exploratory analysis, creation of goodness of fit (GOF) plots, and summary statistics. Campsis (version 1.1.0), an R-based software application provided by Calvagone, was used for simulations.

The NONMEM version 7.5.0 was used with the GNU Fortran (GCC) compiler version 9.3.0, running on a Ubuntu 20.04 LTS system with Linux kernel version 5.4.0-72-generic on Intel Xeon E5-2640 v3 @ 8 HT 2.60 GHz, using VMWare ESXi 7.0u1 platform with fault tolerance, hosting virtual management server with 8 vCPU; and calculation nodes with Intel Xeon E5-2680 v4 14HT 2.40 GHz. Fortran compiler optimisation options were:-static-w-O3-ffast-math. All software was used in a validated environment.

In the mixed effects modelling the population parameters are categorised as follows: Fixed effects parameters, θ (theta), are used for the structural population parameters (e.g. Ka, CL, V, F, etc.), as well as certain parameters relating the population parameters to the physiological or demographic variables (covariates) included in the analysis. Random effects parameters, n (eta), are used for describing inter-individual variability (IIV) in the fixed effects parameters, and can also be used to describe inter-occasion variability (IOV) if appropriately included in the model. Eta parameters are assumed to be normally distributed random variables with mean zero and variance-covariance matrix Q (omega). The square root of the diagonal elements of this matrix provides the IIV estimates. Off-diagonal elements (if not fixed to zero) are commonly translated to correlations. Random effects parameters, ¿ (epsilon), are used to describe the residual variability. Residual variability includes intra-individual error, analytical error, potential error due to model missspecification and any other error not considered by structural elements such as θ and η. Residual variability parameters are assumed to be normally distributed random variables with mean zero and standard deviation σ (sigma).

Different structural models combined with different residual error models and, if needed, with different estimation methods were applied to identify the most appropriate mixed-effects model. The parameter estimation was performed using the First Order Condition Estimation (FOCE) with the INTERACTION option to account for interactions between n for the inter-individual random effects and ε for the residual random effects. An exponential model was used to describe the inter-individual variability (IIV) of the model parameters, assuming lognormal distributions of the individual pharmacokinetic parameters:

PP mm=PP TTTT×ffηη

where P I is the individual parameter value, P TV is the typical population estimate of that parameter and η is the individual deviation from the population estimate with mean 0 and variance ω2. Correlations between random effect parameters were investigated by estimation of some selected off-diagonal elements of the omega matrix. Inter-occasional variability (IOV) was evaluated for the parameters F and CL/F to potentially account for the variability associated with observations of steady-state PK that are taken at completely distinct visits during the study. Data were modelled using a log-transform both sides (LTBS) approach whereby the residual error was incorporated as an additive error on the log scale. Separate residual error terms were explored between healthy subjects and patients.

Covariate model building is exploratory. Initially the potential relationship between covariates and individual parameters estimates will be explored graphically if the degree of eta-shrinkage in the parameters is reasonably low (<25%). Since the model is aimed to be used for exposure simulations in to children, it was decided to account for the association between body weight and PK parameters according to allometric theory already in the base model. A fixed allometric exponent of 0.75 for clearance and inter-compartment flow (Q) was used while an exponent of 1.0 was used for the relationship between volume and body weight. Automated stepwise covariate modelling (SCM) using a forward addition/backward deletion procedure as implemented in PsN [20] was applied to the base model on the complete analysis dataset. To improve the computation time of the SCM, PsN generates a linearized form of the model applying a Taylor transformation on which then the covariate function are applied. The final model from the SCM procedure was then rerun in its original, untransformed form. The following covariates were tested in the model for their association with CL and relative bioavailability (F): body weight, BMI, age, race (if more than 5% non-white participants), sex, liver/kidney function (AST, ALT, albumin, CRCL, eGFR), disease (equivalent to study). In principle, covariate values at baseline were considered, however, an assessment was made if variations of the covariate over time would was needed to be taken into account in the covariate model as well. Correlations between the covariates was evaluated graphically. If two or more covariates were highly correlated then the most clinically/biologically and practically relevant covariate was tested in the covariate analysis with priority, and the other covariate was discarded or tested only after the more relevant one had been included in the model. For continuous covariates linear or power functions were tested, as shown in the following equations:

$${P}_{i} = {P}_{TV} \times ( 1 + θ \times ( {COVAR}_{i} - {MED}_{COVAR} ) ; linear function$$

$${P}_{i} = {P}_{TV} \times {( \frac{{COVAR}_{i}}{{MED}_{COVAR}} )}^{θ} ; power function$$

where P i is the parameter estimate for the i th patient with the value of the covariate (COVARi) centered or scaled to the typical median of the covariate (MEDCOVAR). θ is the slope (exponent) of the parametercovariate relationship. A dichotomous categorical covariate was included as shown below:
where θ is the coefficient of the parameter-covariate relationship. CATCOVAR takes values of 0 or 1 for the state of the covariate value (absent/present) in a particular individual. During the SCM a forward addition/backward deletion procedure was performed. Generally, during forward selection, covariates were introduced into the model one at a time and only the covariate model relationships that are deemed significant were considered for the next step. The covariate that has the highest statistical significance will be included first, and then other significant covariates from the first step will re-tested. Again, the covariate that is then most significant will be retained. This process will be repeated until there is no further improvement in the model. During backward deletion, covariates will be removed one at a time. The most insignificant parameter will be removed from the model first and the process repeated until no covariate can be deleted without significantly deteriorating the model adjustment. Forward covariate selection was performed using a p-value of p<0.05 (χp=0.05,v=1 2=3.84) as the selection criterion. Subsequently, backwards deletion was performed using a p-value of p<0.01 (χp=0.01,v=12=6.63) as the selection criterion. Covariates included in the model after the covariate search was completed, were further assessed for their clinical relevance and their contribution in explaining the interindividual variability. Clinical relevance was judged on the basis of changes to the predicted AUC at different values of a given covariate (e.g. % change in AUC for a patient with high (low) body weight compared to a patient with median weight). A minor change would indicate that the covariate would likely have limited clinical relevance.

### Results

The clinical effect of a reduction in protein to creatinine ratio as a biomarker of kidney function is generated by the administration of a 120 mg BID dose of repagermanium that leads to a steady state of approximately 15 ug·h/mL (range 5-25) AUC as shown in Table 3.

TABLE 3
Summary statistics of individual steady-state exposure (AUC₂₄ₕᵣ)
for a 240 mg total daily dose in the subjects evaluated in the analysis
Steady-state exposure in adults following 240 mg total daily dose

| | 101 | 202 | 203 |
|---|---|---|---|
| N (%) | 13 (20.3%) | 8 (12.5%) | 43 (67.2%) |
| Clearance (L/h) | | | |
| Median | 40.2 | 18.0 | 20.7 |
| Range | 24.6-60.1 | 4.6-36.5 | 5.8-47.6 |
| F(rel. bioavailability) | | | |
| Median | 1.6 | 1.1 | 1.0 |
| Range | 0.7-3.7 | 0.9-1.7 | 0.4-1.9 |
| AUC24h (ug · h/mL) | | | |
| Median | 9.8 | 16.7 | 11.8 |
| Range | 5.5-24.9 | 7.8-45.2 | 3.9-47.7 |

This specific dose and exposure of repagermanium is required for peak renal efficacy.

### Example 2

ARB-CCR2 Inhibitor Combinations that Inhibit G Protein Activation for the Treatment of Disease where the ARB Potency at the AT₁R-CCR2 Complex is not Lower than the Expected Potency of the ARB for AT₁R Only

CCR2-AT₁R complexes have previously been identified as a target for the treatment of renal diseases. Inhibition of CCR2 pathways as well as AT₁R signalling is required for clinical effectiveness.

The present inventors have previously demonstrated consistently high inhibition of inositol phosphate signalling by the AT₁R/CCR2 heteromer with ARBs. The present inventors have also demonstrated that the CCR2-AT₁R complex results in activation of Gαi proteins as well as recruitment of β-arrestin.

However, it was unknown if ARBs maintain their potency for inhibition of Gαi protein activation or recruitment of B-arrestin when acting at the CCR2-AT₁R complex compared to AT₁R alone. Understanding the ability of ARBs to inhibit AT₁R in the CCR2-AT₁R complex is important as it would be expected to influence clinical effectiveness.

For example, the present inventors demonstrated that AngII inhibits the MCP-1-induced activation of Gα1 coupling by CCR2-AT₁R complexes. Blockade of this AngII mediated responses using an ARB, would be expected to remove this inhibition of MCP-1-induced Gαi1 signalling.

The present experiment assessed the potency of ARBs at AT₁R only, versus the potency of ARBs at co-stimulated CCR2-AT₁R complexes in a Gαi protein dissociation assay.

### Methods

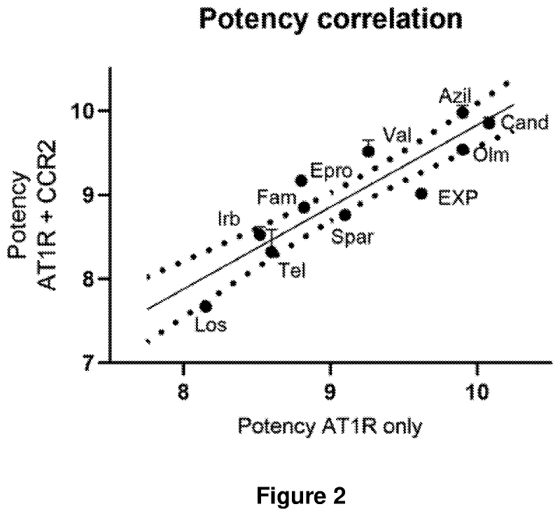

To establish if ARBs maintain their potency for inhibition of Gαi protein activation the potencies of ARBs at AT₁R only were correlated with the potency of ARBs at co-stimulated CCR2-AT₁R complexes obtained from a Gαi protein dissociation assay (FIG. 2).

The potencies of ARBs at AT₁R only were obtained from publicly available sources such as the IUPHAR guide to pharmacology database and/or Michel et al (2013). The potency of ARBs at the co-stimulated CCR2-AT₁R complexes were experimentally derived as outlined below.

HEK293FT cells were transiently transfected by the plasmids coding for Gαi1-Nluc and CCR2-YFP haemagglutinin (HA)-tagged AT₁R. 48 h post-transfection, cells were incubated with substrate for 1 hour before cells were pre-incubated with various concentrations of ARBs (candesartan cilexetil, losartan, telmisartan, irbesartan, eprosartan, EXP3174, valsartan, olmesartan medoximil, olmasartan, azilsartan kamedoxomil or candesartan) for 30 minutes. Cells were then treated with 100 nM AngII and 100 nM MCP-1 or vehicle and ligand induced BRET measured by measuring sequential light emissions at 400-475 nm and 520-540 nm. The BRET signal was calculated by subtracting the ratio of 520-540 nm emission over 400-475 nm emission for a vehicle-treated cell sample from the same ratio for a second aliquot of the same cells treated with ligand (ligand-induced BRET).

GraphPad Prism (v9.3.1) was then used plot ligand-induced BRET verses ARB concentration and potency (pIC₅₀-values) were obtained by fitting a sigmoidal concentration response curve using non-linear regression function of GraphPad Prism (v9.3.1).

To calculate the potency of sparsentan HEK293FT cells were transiently transfected by the plasmids coding for Gαi1-Rluc8, CCR2-YFP and haemagglutinin (HA)-tagged AT₁R. 48 h post-transfection, cells were incubated with substrate for 2 hours before cells were pre-incubated with sparsentan (1 μM) and filtered light emissions were sequentially measured for 1 s in each of the ‘donor wavelength window’ (410-490 nm) and ‘acceptor wavelength window’ (520-620 nm) for 30 minutes. Cells were then stimulated with 10 nM MCP-1 (CCL2) and various concentrations of AngII and filtered light emissions measured for a further 1 hour.

GraphPad Prism (v9.3.1) was then used plot ligand-induced BRET verses AngII concentration in the absence and presence of sparsentan (1 μM) sigmoidal concentration response curves were fit to the data using non-linear regression function of GraphPad Prism (v9.3.1) and sparsentan affinity estimates (pKB) was calculated as pKB as described in Furchgott ((1972) The classification of adrenoceptors (adrenergic receptors). An evaluation from the standpoint of receptor theory, in Catecholamines, Handbook of Experimental Pharmacology (Blaschko H and Muscholl E eds) Vol 33, pp 283-335).

The potency of ARBs at co-stimulated CCR2-AT₁R complexes was then compared to the potency of ARBs at AT₁R using the linear regression function of GraphPad Prism (v9.3.1). The data in FIG. 2 was then analysed using robust linear regression and outlier removal (ROUT) coefficient set to Q=1 to identify outliers with lower-than-expected potency.

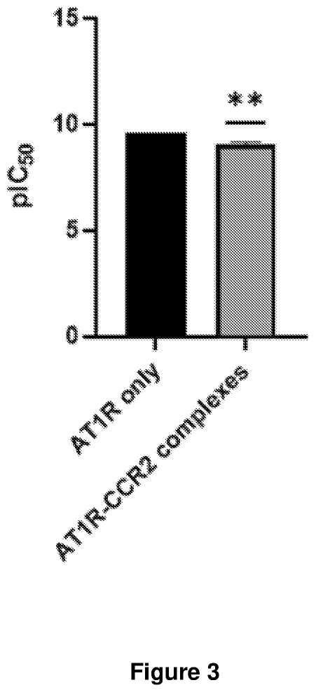

ARBs with potencies at the CCR2-AT₁R complex compared to the AT₁R only that were ≥3-fold lower than expected were considered pharmacologically relevant and analysed by a one-sample t-test to assess statistical significance (FIG. 3).

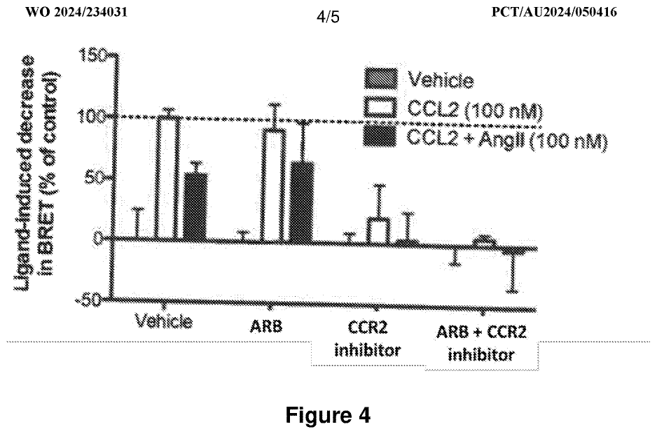

FIG. 4 shows the effect of a combination of an ARB and a CCR2 pathway inhibitor on Gαi1 coupling to the CCR2-AT₁R complex as measured by Ligand-induced BRET. HEK293FT cells were transiently transfected by the plasmids coding for Gαi1-Rluc8 and CCR2-YFP in the presence of haemagglutinin (HA)-tagged AT₁R. 48 h post-transfection, cells were used to generate the agonist-induced BRET signal data in live cells stimulated with 100 nM MCP-1 (CCL2) or 100 nM MCP-1 (CCL2) and 100 nM AngII in the presence of irbesartan, a CCR2 pathway inhibitor or irbesartan and a CCR2 pathway inhibitor.

Upon addition of BRET substrate, BRET detection was carried out in live cells by measuring sequential light emissions at 400-475 nm and 520-540 nm before and after agonist addition. The BRET signal was calculated by subtracting the ratio of 520-540 nm emission over 400-475 nm emission for a vehicle-treated cell sample from the same ratio for a second aliquot of the same cells treated with ligand (ligand-induced BRET). Activation of CCR2 resulted in a decrease in BRET ratio, indicating that activation resulted in a change of conformation of a Gαi1 protein pre-assembled with CCR2. Therefore, the data have been presented as ‘Ligand induced Decrease in in BRET (% of Control)’ such that the change in BRET signal observed with 100 nM MCP-1 is designated 100%, and no change in BRET signal being designated 0%.

### Results

AT₁R only potency values were obtained from IUPHAR guide to pharmacology Alexander et al, (2021) The Concise Guide to Pharmacology 2021/22 Br J Pharmacol. 2021. 178 Suppl 1: S1-S513 and/or Michel et al (2013) A Systematic Comparison of the Properties of Clinically Used Angiotensin II Type 1 Receptor Antagonists Pharmacol Rev 65:809-848These values represent the expected potency of ARBs at co-stimulated CCR2-AT₁R complexes. Experimental potency values are reported as pIC₅₀ or pKB values. pIC50-values are the negative log of the molar concentration of the ARB needed to inhibit the change in BRET mediated by the co-stimulated CCR2-AT₁R complex by 50%. Experimental potency values reported as pKB are the negative log of the molar concentration which at equilibrium would occupy 50% of the receptors in the absence of agonist.

Comparison of ARB potencies (FIG. 2) and Table 4 at AT₁R only and co-stimulated CCR2-AT₁R complexes found a good linear regression R²=0.97 indicating a good correlation between the potency of ARBs for co-stimulated CCR2-AT₁R complexes and AT₁R only.

This data demonstrates that the potency of most ARBs when acting the CCR2-AT₁R complex is within 3.5-fold of the ARB potency at the AT₁R receptor only. This supports the use of ARBs that do not have a reduction in potency when targeting CCR2-AT₁R complexes for the treatment of disease.

In contrast, outlier analysis identified that EXP3174 had lower-than-expected potency for inhibition of Gαi protein activation mediated by co-stimulated the CCR2-AT₁R complexes compared to the potency of EXP3147 at AT₁R only.

The potency of EX3147 at CCR2-AT₁R complexes was found to be 4.07-fold lower and statistically different (FIG. 3; p<0.01, one-sample t-test) compared to the AT₁R only. This data demonstrates that the potency of ARBs acting at CCR2-AT₁R complexes is not obvious and cannot be assumed to be the same as the potency for AT₁R only. ARBs such as EXP3174 that have reduced potency for CCR2-AT₁R complexes are not preferred for targeting CCR2-AT₁R complexes for the treatment of disease and would be expected to have reduced efficacy.

FIG. 4 demonstrates the change in ligand induced BRET following stimulation of cells expressing CCR2-AT₁R complexes with vehicle, CCL2 (100 nM) or, CCL2 (100 nM) and AngII (100 nM). Gαi1 protein activation in cells expressing CCR2-AT₁R co-stimulated with CCL2 (100 nM) and AngII (100 nM) is inhibited by a combination of an ARB and a CCR2 pathway inhibitor. It would be anticipated that a loss of ARB potency at the CCR2-AT₁R complex compared to AT₁R only would influence the inhibitory effectiveness.

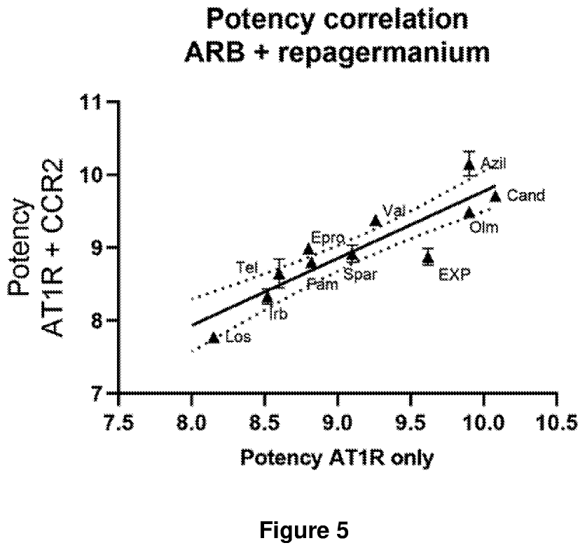

FIG. 5 and Table 4 demonstrates the anticipated potency of ARBs in combination with the CCR2 pathway inhibitor repagermanium for inhibition of Gαi1 protein activation in cells expressing CCR2-AT₁R co-stimulated with CCL2 (100 nM) and AngII (100 nM).

TABLE 4
Inhibitory potencies or affinities (pIC₅₀/pKb) of ARBs at AT₁R
only or co-stimulated AT₁R+ CCR2 complexes

| | | | | | AT₁R + CCR2 + | |
|---|---|---|---|---|---|---|
| | AT₁R only | | AT₁R-CCR2 | | repagermanium | |
| | Fold | | Fold | | Fold | |
| | decrease in | Potency¹ | decrease in | Potency² | decrease in | Potency³ |
| ARB | potency | (pIC₅₀/pKb) | potency | (pIC₅₀/pKb) | potency | (pIC₅₀/pKb) |
| Azilsartan | 1.00 | 9.9 | 0.85 | 9.97 | 0.56 | 10.15 |
| Candesartan | 1.00 | 10 | 1.41 | 9.85 | 1.95 | 9.71 |
| Candesartan | — | — | — | 8.72 | — | 8.63 |
| cilexetil | | | | | | |
| Eprosartan | 1.00 | 8.8 | 0.44 | 9.16 | 0.66 | 8.98 |
| EXP3174 | 1.00 | 9.62 | 4.07** | 9.01 | 5.62** | 8.87 |
| Irbesartan | 1.00 | 8.52 | 1.00 | 8.52 | 1.55 | 8.33 |
| Losartan | 1.00 | 8.05 | 2.45 | 7.66 | 1.91 | 7.77 |
| Olmesartan | 1.00 | 9.9 | 2.34 | 9.53 | 2.57 | 9.49 |
| OlmesartanM | — | — | — | 9.73 | — | 9.56 |
| Sparsentan | 1.00 | 9.1 | 2.19 | 8.76# | 1.51 | 8.92 |
| Telmisartan | 1.00 | 8.61 | 1.95 | 8.32 | 0.93 | 8.64 |
| Valsartan | 1.00 | 9.26 | 0.56 | 9.51 | 0.76 | 9.38 |
| Fimasartan | 1.00 | 8.82^{$} | 0.93 | 8.85 | 1.05 | 8.80 |

1. From IUPHAR guide to pharmacology and/or Michel et al (2013). 2. ARB potencies (pIC₅₀) for inhibition of Ga-protein dissociation from CCR2/Luc-AT1R complexes co-stimulated with AngII and CCL2. 3. Prophetic ARB potency for inhibition of Gα-protein dissociation from CCR2/Luc-AT1R complexes co-stimulated with AngII and CCL2 in the presence of repagermanium. \hat{ }; Fimasartan data is prophetic. $, data from Choung et al, 2018 Discovery of the bifunctional modulator of angiotensin II type 1 receptor (AT1R) and PPARγ derived from the AT1R antagonist, Fimasartan. Bioorganic & Medicinal Chemistry Letters (28), 19:3155-3160 #Sparsentan affinity (pKb) at AT1R-CCR2 complex. **, p<0.01 one-sample t-test, significant decrease in potency compared to AT1R only.

1. (canceled)

2. (canceled)

3. A therapeutic formulation adapted to deliver daily dose of:
a) a therapeutically effective amount of repagermanium or propagermanium; and/or
b) a therapeutically effective amount of an AT₁R blocker that has an affinity for the CCR2-AT₁R complex that is the same or greater than the AT₁R blocker's affinity for AT₁R only.

4. The formulation of claim 3 wherein the daily dose of repagermanium or propagermanium is from about 200 mg to 280 mg.

5. The formulation of claim 3, wherein the AT₁R blocker is chosen from candesartan, eprosartan, irbesartan, losartan, olmesartan, telmisartan, valsartan, fimasartan or sparsentan.

6. The formulation of claim 3, wherein the daily dose of the AT₁R blocker is from 2 mg to 800 mg.

7. (canceled)

8. (canceled)

9. (canceled)

10. (canceled)

11. (canceled)

12. A method for treatment, amelioration or prevention of a kidney disease, said method comprising the step of administering to a subject:
a) a therapeutically effective amount of repagermanium or propagermanium per day; and/or
b) a therapeutically effective amount of an AT₁R blocker that has an affinity for the CCR2-AT₁R complex that is the same or greater than the AT₁R blocker's affinity for AT₁R only.

13. (canceled)

14. (canceled)

15. (canceled)

16. (canceled)

17. (canceled)

18. (canceled)

19. (canceled)

20. (canceled)

21. (canceled)

22. (canceled)

23. A kit for the treatment or prevention of a kidney disease in a subject, said kit comprising:
a) a therapeutically effective amount of repagermanium or propagermanium; and/or
b) a therapeutically effective amount of an AT₁R blocker that has an affinity for the CCR2-AT₁R complex that is the same or greater than the AT₁R blocker's affinity for AT₁R only; and
c) instructions for use.

24. The method of claim 12, wherein the repagermanium or propagermanium is provided at a daily dose of from about 200 mg to 280 mg repagermanium or propagermanium.

25. The method of claim 12, wherein the AT₁R blocker is chosen from the list consisting of: candesartan, eprosartan, fimasartan, irbesartan, losartan, olmesartan, telmisartan, valsartan, fimasartan and sparsentan.

26. The method of claim 12, wherein the subject is a human mammal.

27. The method of claim 12, wherein the kidney disease is a kidney disease associated with proteinuria.

28. The method of claim 12, wherein the kidney disease is chosen from focal segmental glomerulosclerosis (FSGS, fibrotic disorders in the kidney, Immunoglobulin A nephropathy (IgAN), Alport's Syndrome, chronic kidney disease including chronic kidney disease caused by diabetic nephropathy, diabetic renal insufficiency, non-diabetic renal insufficiency, and renal failure conditions.

29. The method of claim 12, wherein the AT₁R blocker is not EXP3174.

30. The method of claim 28, wherein the FSGS is chosen from idiopathic FSGS, secondary FSGS, or genetic FSGS.

31. The method of claim 28, wherein the renal failure conditions are chosen from diabetic nephropathy, glomerulonephritis, scleroderma, glomerular sclerosis, proteinuria of primary renal disease, or renal vascular hypertension.
